(19) United States
(12) Patent Application Publication
(10) Pub. No.: US 2012/0109001 A1
(43) Pub. Date: May 3, 2012

(54) SENSING IN AN IMPLANTABLE DEVICE IN THE PRESENCE OF AN INTERFERING SIGNAL USING LEAD IMPEDANCE MEASUREMENTS

(75) Inventor: Michael L. Ellingson, St. Louis Park, MN (US)

(73) Assignee: Medtronic, Inc.

(21) Appl. No.: 12/915,252

(22) Filed: Oct. 29, 2010

(51) Int. Cl. A61B 5/053; A61N 1/08

(52) U.S. Cl. 600/547; 607/28

(57) ABSTRACT

A device to which an implantable lead is coupled may be configured to perform one or more impedance measurements subsequent to a sensed physiological event to determine whether the sensed physiological event is possibly associated with noise induced by an interfering signal. Interfering signals, such as electromagnetic fields generated by an external source, may induce voltages or currents on conductors of the implantable lead, which will have an effect on lead impedance measurements. By measuring one or more impedances associated with the lead during a time window that substantially coincides with the sensed physiological event, the device may determine whether the sensed events may have resulted from interference. The device may determine whether to further adjust operation of the IMD based on the classification of the sensed physiological event and a classification of one or more previously sensed physiological events.

### TECHNICAL FIELD

The disclosure relates generally to implantable medical devices and, in particular, to sensing by implantable medical devices in the presence of an interfering signal from an external source.

### BACKGROUND

A wide variety of medical systems are implanted within patients to provide a therapy to and/or monitor a physiologic condition of a patient. These implantable medical systems may include an implantable medical device (IMD) and one or more implantable medical leads to deliver therapy to or monitor conditions of a number of organs, nerves, muscles or tissues of the patient, such as the heart, brain, stomach, spinal cord, pelvic floor or the like.

Occasionally, patients that have implantable medical systems may benefit from a medical procedure that may generate an interfering signal. For example, a patient may benefit from a magnet resonance image being taken of a particular area of his or her body. Magnetic resonance imaging (MRI) is a technique for imaging portions of the body of the patient for purposes of medical diagnosis. During an MRI procedure, the patient is exposed to magnetic and radio frequency (RF) fields to obtain images of a portion of the body. In particular, the patient is exposed to a strong static (i.e., non-varying) magnetic field that is typically always present around the MRI device whether or not a procedure is in progress. In the presence of the strong static magnetic field, a number of gradient (i.e., time-varying) magnetic fields and RF fields are applied during the MRI procedure to obtain the desired images. The magnitude, frequency or other characteristic of the various fields applied during the MRI procedure may vary based on the type of device producing the fields or the type of scan being performed.

Exposure of the implantable medical system to the various fields generated by the MRI device may result in undesirable operation of the implantable medical system. In some instances, the gradient magnetic fields or the RF fields may induce voltages or currents on the leads of the implantable medical system. The voltages or currents induced on the leads may interfere with the ability of the IMD to properly sense cardiac signals of the heart of the patient. For example, voltages or currents induced on the lead by the gradient magnetic or RF fields may cause the IMD to incorrectly sense a cardiac signal when one is not present or to fail to sense a cardiac signal when one is present. Such interference may result in the IMD delivering therapy when it is not desired or withholding therapy when it is desired.

### SUMMARY

This disclosure describes techniques to improve operation of an implantable medical system during exposure to an interfering signal. Interfering signals, such as electromagnetic fields generated by an external source, may induce voltages or currents on conductors of an implantable medical lead. The induced voltages or currents will have an effect on lead impedance measurements, which rely on inducing and measuring currents and voltages on the leads.

As such, an IMD to which the lead is coupled may be configured to perform one or more impedance measurements subsequent to each sensed physiological event to determine whether the sensed physiological event is possibly associated with noise induced by the interfering signal. By measuring one or more impedances associated with the lead during a time window that substantially coincides with the sensed physiological event, IMD 14 may determine whether the sensed events may have resulted from interference. In particular, IMD 14 may analyze the one or more impedance measurements for irregularities that may be caused by the interfering signal, such as unexpected increase(s) or decrease(s) in lead impedance during the impedance measurement(s). Based on the analysis, IMD 14 may classify the sensed physiological event as an actual cardiac event or a potential oversensed cardiac event caused by an interfering signal. IMD 14 may determine whether to further adjust operation of IMD 14 based on the classification of the sensed physiological event and a classification of one or more previous sensed physiological events.

In one example, this disclosure is directed to an implantable medical device comprising a sensing module that senses a physiological event and a lead impedance module that measures one or more lead impedances subsequent to the sensing module sensing the physiological event and classifies the sensed physiological event as an actual physiological event or a possible oversensed physiological event based on the one or more lead impedance measurements.

In another example, this disclosure is directed to a method comprising sensing a physiological event, measuring one or more lead impedances subsequent to sensing the physiological event, and classifying the sensed physiological event as an actual physiological event or a possible oversensed physiological event based on the one or more lead impedances.

In a further example, this disclosure is directed to an implantable medical device comprising means for sensing a physiological event, means for measuring one or more lead impedances subsequent to sensing the physiological event, and means for classifying the sensed physiological event as an actual physiological event or a possible oversensed physiological event based on the one or more lead impedances.

In another example, this disclosure is directed to a computer-readable medium comprising instructions that, when executed by a processor, cause the processor to sense a physiological event, measure one or more lead impedances subsequent to sensing the physiological event, and classify the sensed physiological event as an actual physiological event or a possible oversensed physiological event based on the one or more lead impedances.

This summary is intended to provide an overview of the subject matter described in this disclosure. It is not intended to provide an exclusive or exhaustive explanation of the techniques as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF THE DRAWINGS

FIG. 1 is a conceptual diagram illustrating an environment in which an implantable medical system is exposed to an interfering signal from an external source.

FIG. 2 is a conceptual diagram illustrating the implantable medical system of FIG. 1 in more detail.

FIG. 3 is a functional block diagram of an example configuration of components of an IMD of the implantable medical system.

FIG. 4 is a flow diagram illustrating example operation of an IMD using lead impedance measurements to enhance sensing.

### DETAILED DESCRIPTION

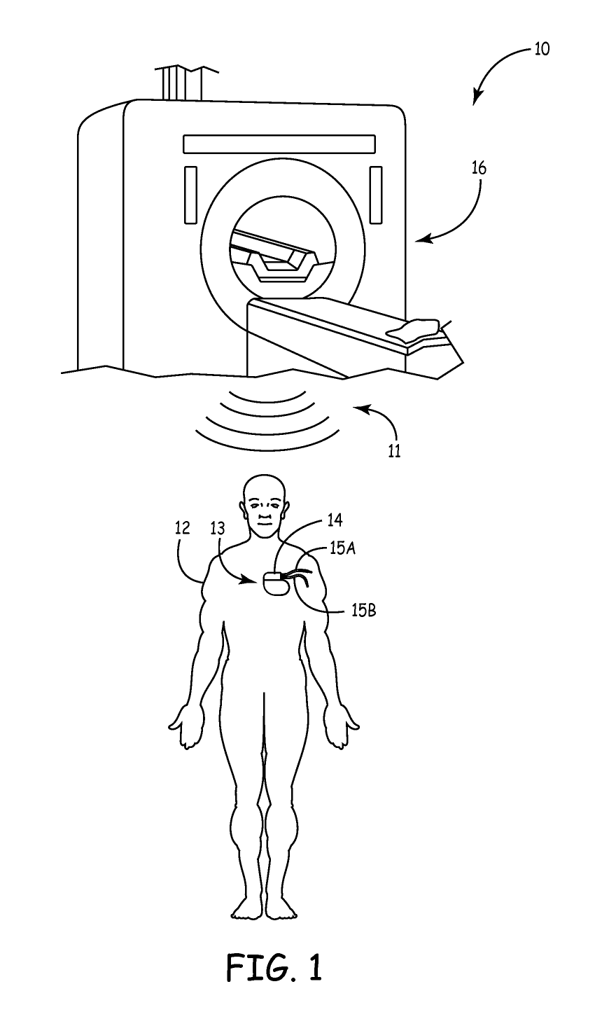

FIG. 1 is a conceptual diagram illustrating an environment 10 in which an implantable medical system 13 is exposed to an interfering signal 11 from an external source. Implantable medical system 13 of FIG. 1 includes an implantable medical device (IMD) 14 and one or more leads (e.g., leads 15A and 15B, collectively “leads 15”) implanted within patient 12. IMD 14 and leads 15 are adapted to provide therapy to and/or to monitor one or more physiological parameters of patient 12. The techniques, however, are not limited to devices implanted within patient 12. For example, the techniques may be used in conjunction with an external medical system that senses one or more parameters of patient 12 and is adversely affected by interfering signal 11.

Environment 10 includes an external energy source 16 that generates interfering signal 11 to which implantable system 13 is exposed. In the example illustrated in FIG. 1, the external energy source is an MRI device 16. Although the techniques of this disclosure are described with respect to interfering signal 11 generated by MRI device 16, the techniques may be used to enhance sensing by IMD 14 within environments in which other types of interfering signals are present. For example, IMD 14 may operate in accordance with the techniques of this disclosure in environments in which interfering signal 11 is generated by other sources, such as an electrocautery device, diathermy device, ablation device, radiation therapy device, electrical therapy device, magnetic therapy device, radio frequency identification (RFID) readers, or any other environment with devices that radiate energy to produce magnetic, electromagnetic, electric or other interfering energy fields.

MRI device 16 uses magnetic and RF fields to produce images of body structures for diagnosing injuries, diseases and/or disorders. In particular, MRI device 16 generates a static magnetic field, gradient magnetic fields and RF fields. The static magnetic field is a non-varying magnetic field that is typically present around MRI device 16 whether or not an MRI scan is in progress. Gradient magnetic fields are time-varying magnetic fields that are typically only present while the MRI scan is in progress. RF fields are pulsed RF fields that are also typically only present while the MRI scan is in progress. The magnitude, frequency or other characteristic of interfering signal 11 may vary based on the type of MRI device producing the field. A 1.5 Tesla MRI device, for example, generates a static magnetic field at approximately 15,000 Gauss, generates gradient magnetic fields up to approximately 45 mT/m at 200 T/m/s, and generates RF pulses at approximately 64 MHz.

Some or all of the various types of fields produced by MRI device 16 may interfere with the operation of IMD 14. In other words, one or more of the various types of fields produced by MRI device 16 may make up interfering signal 11. For example, the gradient magnetic fields and RF fields produced by MRI device 16 may interfere with sensing by IMD 14. In particular, the gradient magnetic fields and RF fields produced by MRI device 16 may induce voltages or currents on implantable leads 15 coupled to IMD 14. The induced voltages or currents on leads 15 distort the sensed electrical cardiac signals, which may result in IMD 14 detecting physiological signals that are not present, a phenomena s sometimes referred to as oversensing. The oversensing caused by interfering signal 11 may in turn cause IMD 14 to deliver undesired therapy or withhold desired therapy.

This disclosure describes techniques to improve sensing during exposure to interfering signal 11. IMD 14 may be configured into an MRI-compatible operating mode prior to or immediately subsequent to entering environment 10. The MRI-compatible operating mode may include changes to one or more operating parameters of IMD 14 that provide enhanced sensing of physiological signals. In the MRI-compatible operating mode, for example, IMD 14 may perform one or more impedance measurements subsequent to each sensed physiological event to determine whether the sensed physiological event is possibly associated with noise. Because electromagnetic fields such as interfering signal 11 may affect the one or more impedance measurements, IMD 14 may analyze the one or more impedance measurements to classify the sensed physiological event is an actual physiological event or a possible oversensed event due to noise from interfering signal 11. IMD 14 may determine whether to further adjust operation of IMD 14 based on classifications made over a number of consecutive sensed physiological events.

IMD 14 may be automatically configured into the MRI-compatible operating mode in response to detecting one or more conditions indicative of the presence of MRI device 16, e.g., existence of a strong magnetic field detected by a Hall sensor or other magnetic sensor. In other instances, IMD 14 is manually programmed into the MRI-compatible operating mode prior to entering environment 10. For example, an external device (not illustrated) may wirelessly communicate with IMD 14 to send one or more commands that cause IMD 14 to transition to the MRI-compatible operating mode.

Although the techniques of this disclosure are described in the context of environment 10 including an MRI device 16 as the external source, the techniques may be used in other environments in which the standard sampling configuration does not allow for accurate interpretation of cardiac electrical signals due to interfering signals, including but not limited environments during electrocautery procedures, diathermy procedures, ablation procedures, radiation therapy procedures, electrical therapy procedures, and magnetic therapy procedures. In other instances, the techniques may be used to assess the integrity of a lead or a lead/device interface when oversensing is suspected. For example, to detect if a lead is not securely connected to a device or if a lead has experienced a conductor fracture, both of which may result in the device misinterpreting make/break connections as physiological signals. In yet other instances, the techniques may be applied during all sensed events.

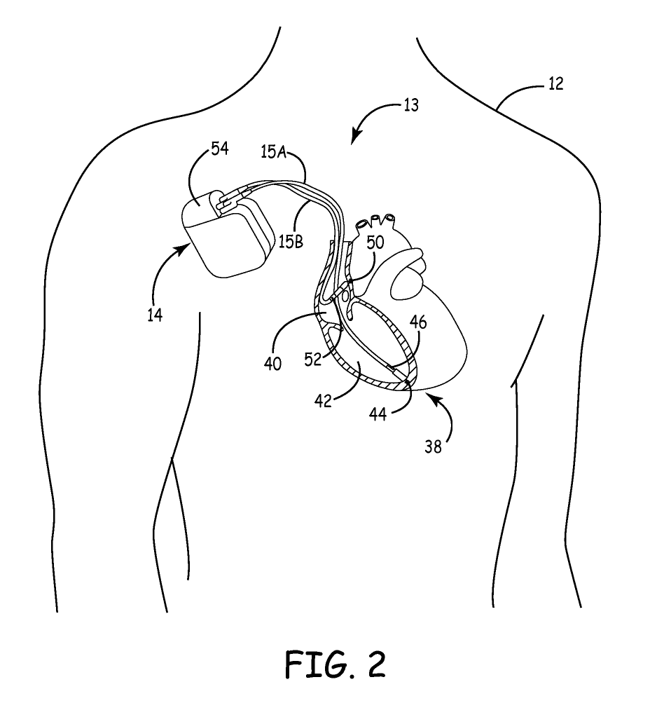

FIG. 2 is a conceptual diagram illustrating implantable medical system 13 in more detail. Implantable medical system 13 includes an IMD 14 and leads 15 that extend from IMD 14. In the example illustrated in FIG. 2, IMD 14 is an implantable cardiac device that senses electrical activity of a heart 38 of patient 12 and provides electrical stimulation therapy to heart 38 of patient 12. The electrical stimulation therapy to heart 38, sometimes referred to as cardiac rhythm management therapy, may include pacing, cardioversion, defibrillation and/or cardiac resynchronization therapy (CRT).

In the illustrated example, lead 15A is a right ventricular (RV) lead that extends through one or more veins (not shown), the superior vena cava (not shown), and right atrium 40, and into right ventricle 42 of heart 38. Lead 15A includes electrodes 44 and 46 located along a distal end of lead 15A. In the illustrated example, lead 15B is a right atrial (RA) lead that extends through one or more veins and the superior vena cava, and into the right atrium 40 of heart 38. Lead 15B includes electrodes 50 and 52 located along a distal end of lead 15B.

Electrodes 44 and 50 may take the form of extendable helix tip electrodes mounted retractably within an insulative electrode head (not shown) of respective leads 15. Electrodes 46 and 52 may take the form of ring electrodes. In other embodiments, electrodes 44, 46, 50 and 52 may be other types of electrodes. For example, electrodes 44, 46, 50 and 52 may all be ring electrodes located along the distal end of the associated leads 15. Additionally, either or both of leads 15 may include more than two electrodes or only a single electrode.

Each of the electrodes 44, 46, 50 and 52 may be electrically coupled to a respective conductor within the body of its associated lead 15. The respective conductors may extend from the distal end of the lead to the proximal end of the lead and couple to circuitry of IMD 14. For example, leads 15 may be electrically coupled to a stimulation module, a sensing module, or other modules of IMD 14 via connector block 54. In some examples, the proximal ends of leads 15 may include electrical contacts that electrically couple to respective electrical contacts within connector block 54. In addition, in some examples, leads 15 may be mechanically coupled to connector block 54 with the aid of set screws, connection pins or another suitable mechanical coupling mechanism.

Electrodes 44, 46, 50 and 52 may be used to sense cardiac electrical signals attendant to the depolarization and repolarization of heart 38. The cardiac electrical signals are conducted to IMD 14 via one or more conductors of respective leads 15. IMD 14 may use any combinations of the electrodes 44, 46, 50, 52 or the housing electrode for unipolar or bipolar sensing. As such, the configurations of electrodes used by IMD 14 for sensing and pacing may be unipolar or bipolar depending on the application. IMD 14 may analyze the sensed signals to monitor a rhythm of heart 38 or detect an abnormal arrhythmia of heart 38, e.g., tachycardia, bradycardia, fibrillation or the like. In some instances, IMD 14 provides electrical stimulation therapy based on the cardiac signals sensed within heart 38. For example, IMD 14 may trigger or inhibit delivery of the electrical stimulation therapy based on the sensed cardiac signals. In other words, the electrical stimulation therapy may be responsive to the sensed events.

As described above with respect to FIG. 1, exposure of IMD 14 to an interfering signal 11 may introduce noise on the signals received by the sensing components of IMD 14. This noise may cause IMD 14 to oversense cardiac events, i.e., inappropriately detect cardiac events not actually present, which in turn may cause IMD 14 to deliver undesired therapy or withhold desired therapy. IMD 14 may be configured to an MRI-compatible operating mode in which IMD 14 conducts one or more impedance measurements immediately subsequent to detection of a sensed cardiac event to differentiate between actual cardiac events and possible oversensed events caused by the noise.

The configuration of implantable medical system 13 illustrated in FIGS. 1 and 2 is merely an example. In other examples, implantable medical system 13 may include more or fewer leads extending from IMD 14. For example, IMD 14 may be coupled to three leads, e.g., a third lead implanted within a left ventricle of heart 38. In another example, IMD 14 may be coupled to a single lead that is implanted within either an atrium or ventricle of heart 38. As such, IMD 14 may be used for single chamber or multi-chamber cardiac rhythm management therapy.

In addition to more or fewer leads, each of the leads may include more or fewer electrodes. In instances in which IMD 14 is used for therapy other than pacing, e.g., defibrillation or cardioversion, the leads may include elongated electrodes, which may, in some instances, take the form of a coil. IMD 14 may deliver defibrillation or cardioversion shocks to heart 38 via any combination of the elongated electrodes and housing electrode. As another example, implantable medical system 13 may include leads with a plurality of ring electrodes, e.g., as used in some implantable neurostimulators.

The techniques of this disclosure are described in the context of cardiac rhythm management therapy for purposes of illustration. The techniques of this disclosure, however, may be used to operate an IMD that provides other types of electrical stimulation therapy. For example, the IMD may be a device that provides electrical stimulation to a tissue site of patient 12 proximate a muscle, organ or nerve, such as a tissue proximate a vagus nerve, spinal cord, brain, stomach, pelvic floor or the like. As such, description of these techniques in the context of cardiac rhythm management therapy should not be limiting of the techniques as broadly described in this disclosure.

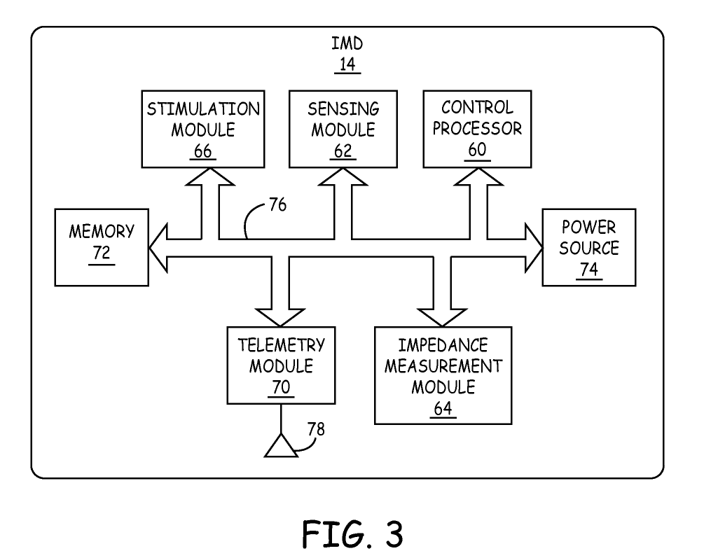

FIG. 3 is a functional block diagram of an example configuration of components of IMD 14. In the example illustrated by FIG. 3, IMD 14 includes a control processor 60, sensing module 62, impedance measurement module 64, stimulation module 66, telemetry module 70, memory 72 and power source 74. The components of IMD 14 are illustrated as being interconnected by a data bus 76, but may be connected by one or more direct electrical connections in addition to or instead of data bus 76.

One or more electrodes 44, 46, 50, or 52 (or the housing electrode) senses electrical signals attendant to the depolarization and repolarization of heart 38. In this manner, the electrodes may be viewed as one example of a sensor. However, the techniques of this disclosure may be used with signals received from other types of sensors. The electrical signals sensed by electrodes 44, 46, 50 or 52 are conducted to sensing module 62 via one or more conductors of leads 15. In other instances, leads 15 may include one or more sensors dedicated for sensing. In further examples, sensing module 62 is coupled to one or more sensors that are not included on leads 15, e.g., via a wired or wireless coupling. Other types of sensors besides electrodes may include, but are not limited to, pressure sensors, accelerometers, flow sensors, blood chemistry sensors, activity sensors or other types of physiological sensors.

Sensing module 62 includes sensing components used to process signals received from the one or more sensors. The components of sensing module 62 may be analog components, digital components or a combination thereof. Sensing module 62 may include multiple sensing channels each having associated sensing components. Each sensing channel may, for example, include one or more sense amplifiers, filters, rectifiers, threshold detectors, analog-to-digital converters (ADCs) or the like. Sensing module may detect cardiac events, such as atrial or ventricular polarizations, and provide indications of the occurrences of such events to processor 60. Sensing module 62 may, for example, compare processed signals to a threshold and detect a cardiac event, e.g., the existence of P- or R-waves when the processed signal exceeds the threshold.

Processor 60 may control stimulation module 66 to provide electrical stimulation therapy based on the cardiac events extracted from the sensed signals. Processor 60 may inhibit stimulation module 66 to withhold delivery of electrical stimulation to heart 38 upon sensing a cardiac event associated with spontaneous depolarization. In another example, processor 60 may trigger stimulation module 66 to deliver electrical stimulation to heart 38 upon sensing a cardiac event associated with spontaneous depolarization. In this manner, the electrical stimulation therapy may be responsive to the sensed events.

Stimulation module 66 delivers electrical stimulation to heart 38 via one or more of electrodes 44, 46, 50, 52 and/or the housing electrode. Stimulation module 66 is electrically coupled to electrodes 44, 46, 50 and 52, e.g., via conductors of the respective leads 15, or, in the case of the housing electrode, via an electrical conductor disposed within the housing of IMD 14. Processor 60 controls stimulation module 66 to generate and deliver electrical pacing pulses with the amplitudes, pulse widths, rates, electrode combinations or electrode polarities specified by a selected therapy program. For example, electrical stimulation module 66 may deliver bipolar pacing pulses via ring electrodes 46 and 52 and respective corresponding helical tip electrodes 44 and 50 of leads 15. To this end, stimulation module 66 may include a pulse generator or other components needed to generate electrical stimulation signals. Stimulation module 66 may deliver one or more of these types of stimulation in the form of other signals besides pulses or shocks, such as sine waves, square waves, or other substantially continuous signals. In addition to pacing pulses, stimulation module 66 may, in some instances, deliver other types of electrical therapy, such as defibrillation, and/or cardioversion.

Exposure of IMD 14 to interfering signal 11 may introduce noise on the cardiac signals received by sensing module 62 of IMD 14. Again, the noise may cause IMD 14 to inappropriately detect cardiac events not actually present (i.e., oversense), which may cause IMD 14 to deliver undesired therapy or withhold desired therapy. This disclosure describes techniques to improve sensing during exposure to interfering signal 11. IMD 14 may be configured into an MRI-compatible operating mode prior to or immediately subsequent to entering environment 10. In the MRI-compatible operating mode IMD 14 performs one or more impedance measurements subsequent to each sensed physiological event to determine whether the sensed physiological event is an actual cardiac event.

Electromagnetic fields, such as interfering signal 11, may affect the impedance measurements. In particular, interfering signal 11 may induce voltages or currents on the conductors of leads 15. The induced voltages or currents will have an effect on lead impedance measurements, which rely on inducing and measuring currents and voltages on the leads. As such, impedance measurement module 64 is configured to measure one or more impedances associated with one or more electrical paths of leads 15 immediately subsequent to identifying a sensed event. In one example, impedance measurement module 64 may begin to measure the one or more impedances within milliseconds or even microseconds of identifying the sensed event. By measuring the one or more impedances associated with leads 15 during a time window that substantially coincides with the sensed cardiac events, IMD 14 may determine whether the sensed events may have resulted from MRI-induced interference. IMD 14 may also determine whether to further adjust operation of IMD 14 based on classifications of sensed events as actual sensed events or possible oversensed events made over a series of sensed physiological events.

Impedance measurement module 64 is configured to measure an impedance of an electrical path that includes any combination of electrodes 44, 46, 50, 52, a housing electrode, or any other electrode. In some instances, impedance measurement module 64 may be configured to measure impedances of the same electrical path on which the cardiac event is sensed. For example, if the sensed event was measured using bipolar sensing on lead 15A (i.e., with electrodes 44 and 46), impedance measurement module 64 may obtain the one or more impedance measurements along the electrical path that includes electrodes 44 and 46. In other instances, impedance measurement module 64 may measure the one or more impedances of a different electrical path than the electrical path used to sense the cardiac event. For example, sensing module 62 may perform bipolar sensing using electrodes 44 and 46 to sense the cardiac event and obtain the one or more impedance measurements along the path including tip electrode 44 and the housing electrode. In this example, a different electrical path of the same lead is used for measuring the one or more impedances. In another example, impedance measurement module 64 may obtain the one or more impedance measurements along an electrical path including electrodes of a different lead than the lead on which the sensed event was detected.

Impedance measurement module 64 includes circuitry to generate one or more impedance measurement signals along a selected electrical path and measure the impedances based on the one or more impedance measurement signals. Impedance measurement module 64 may, for example, include a current source that generates current or a voltage source that generates voltage. In the case of a current source, impedance measurement module 64 transmits one or more current bursts through a selected combination of electrodes 44, 46, 50, 52 or housing electrode to generate the one or more impedance measurement signals. In the case of a voltage source, impedance measurement module 64 generates one or more voltages across any combination of electrodes 44, 46, 50, 52 or housing electrode to generate the one or more impedance measurement signals. The impedance measurement signals may, in some examples, be subthreshold pulses.

Impedance measurement module 64 may also include circuitry configured to measure the impedance along the electrical path based on the impedance measurement signals. In instances in which the impedance measurement signals are current bursts, impedance measurement module 64 may include circuitry to measure voltages associated with the electrical path and determine a series of impedances along the electrical path based on the current bursts and the measured voltages. In instances in which the impedance measurement signals are voltages, impedance measurement module 64 may include circuitry to measure currents associated with the electrical path and determine impedances along the electrical path based on the series of voltages and measured currents. The impedances may be determined, for example, by dividing the voltages (either measured or sourced) by the currents (either sourced or measured, respectively) to determine the impedance measurements of the paths.

Impedance measurement module 64 may analyze the one or more impedance measurements measured immediately subsequent to the sensed event to determine whether the sensed event is an actual sensed event or a possible oversensed event caused by MRI-induced noise. As described above, interfering signal 11 from MRI device 16 may affect the measured impedance(s) associated with leads 15. As such, impedance measurement module 64 may analyze the one or more impedance measurements to identify irregularities among any of the measured impedances.

In some instances, impedance measurement module 64 generates a series of impedance measurement signals to measure a plurality of impedances. For example, impedance measurement module 64 may generate a series of four impedance measurement signals and measure the impedance associated with each of the impedance measurement signals. Impedance measurement module 64 may analyze the series of impedance measurements for irregular variations in amplitudes. Impedance measurement module 64 may compare each of the series of impedance measurements to an impedance range based on a history of past lead impedance measurements obtained by IMD 14, e.g., when IMD 14 is not exposed to interfering signal 11. The typical impedance range may, for example, be equal to ±30% of an average impedance value associated with the electrical path. However, other acceptable impedance ranges may be used, such as ±5%, ±10%, ±15%, ±20%, or ±25% of the average impedance value associated with the electrical path. If all or an acceptable portion (e.g., 3 out of 4) of the impedance measurements are within the impedance range, impedance measurement module 64 classifies the sensed cardiac event as an actual cardiac event. If an unacceptable portion of the impedance measurements are within the impedance range, impedance measurement module 64 characterizes the sensed physiological event as a possible oversensed physiological event. In other instances, impedance measurement module 64 may determine if all impedance measurements are within a preprogrammed range of impedances (e.g., 200 Ohms to 3,000 Ohms), e.g., which is not based on past history of lead impedance measurements obtained by IMD 14. In yet other instances, impedance measurement module 64 may determine if the morphology of a series of lead impedance measurements (i.e., amplitude of the impedance measurements over time) matches or correlates with typical morphologies of impedance measurements based on past history.

In other instances, impedance measurement module 64 may generate only a single impedance measurement signal subsequent to each sensed cardiac event. In this case, impedance measurement module 64 may compare the measured impedance value to the typical impedance range based on past history of lead impedance measurements obtained when IMD 14 is not exposed to interfering signal 11 or a predetermined range to classify the event as an actual cardiac event (e.g., if within the respective range) or as a possible oversensed physiological event (e.g., if not within the respective range).

Processor 60 (or impedance measurement module 64 in some instances) may determine whether sensing is reliable based on classifications of a number of consecutive sensed physiological events and adjust operation of the implantable medical device in response to determining that sensing is unreliable. Processor 60 may, for example, determine that sensing is unreliable when three of the last four sensed physiological events are classified as possible oversensed events. As another example, processor 60 may determine that sensing is unreliable when at least two sensed physiological events are classified as possible oversensed events within one second. In response to determining that sensing is unreliable, processor 60 may further adjust operation of IMD 14 by changing sensing parameters, disabling sensing, changing pacing modes to a mode that is not dependent on sensing, withholding or delaying a therapy (e.g., tachyarrhythmia therapy), or the like.

Although shown as separate modules in FIG. 3, in some examples, all or a portion of impedance measurement module 64 may be formed within stimulation module 66, sensing module 62 or processor 60. For example, the circuitry configured to generate the one or more impedance measurement signals may be included within stimulation module 66 and the circuitry configured to measure the impedance may be included within sensing module 62. As another example, the portion of impedance module 64 configured to analyze the one or more impedance measurements associated with a sensed event may be included within processor 60.

Sensing module 62, impedance measurement module 64 and stimulation module 66 are each coupled to conductors that are coupled to one or more electrodes. In some examples, sensing module 62, impedance measurement module 64 and stimulation module 66 may be coupled to the same conductors that are coupled to one or more electrodes. In these examples, sensing module 62, impedance measurement module 64 and stimulation module 66 may utilize a switch module to select which of the available electrodes are used to sense, stimulate, measure impedance, etc.

Control processor 60 may include any one or more of a microprocessor, a controller, a digital signal processor (DSP), an application specific integrated circuit (ASIC), a field-programmable gate array (FPGA), or equivalent discrete or integrated circuitry, including analog circuitry, digital circuitry, or logic circuitry. The functions attributed to control processor 60 herein may be embodied as software, firmware, hardware or any combination thereof.

Memory 72 may include computer-readable instructions that, when executed by control processor 60 or other component of IMD 14, cause one or more components of IMD 14 to perform various functions attributed to those components in this disclosure. Memory 72 may include any volatile, non-volatile, magnetic, optical, or electrical media, such as a random access memory (RAM), read-only memory (ROM), non-volatile RAM (NVRAM), static non-volatile RAM (SRAM), electrically-erasable programmable ROM (EEPROM), flash memory, or any other computer-readable storage media.

The various components of IMD 14 are coupled to power source 74, which may include a rechargeable or non-rechargeable battery. A non-rechargeable battery may be capable of holding a charge for several years, while a rechargeable battery may be inductively charged from an external device, e.g., on a daily or weekly basis. Power source 74 also may include power supply circuitry for providing regulated voltages and/or current levels to power the various components of IMD 14.

Under the control of processor 60, telemetry module 70 may receive downlink telemetry from and send uplink telemetry to programming device 18 with the aid of an antenna 78, which may be internal and/or external to IMD 14. As described above, for example, telemetry module 70 may receive commands from a programmer indicating that IMD 14 should transition to the MRI-compatible operating mode. Telemetry module 70 includes any suitable hardware, firmware, software or any combination thereof for communicating with another device, such as programming device 18. For example, telemetry module 70 may include appropriate modulation, demodulation, encoding, decoding, frequency conversion, filtering, and amplifier components for transmission and reception of data.

The various modules of IMD 14 may include one or more processors, such as one or more microprocessors, DSPs, ASICs, FPGAs, programmable logic circuitry, or the like, that may perform various functions and operations, such as those described herein. Although illustrated in FIG. 3 as separate modules, the functionality attributed to the modules may be performed by common hardware, firmware or software components.

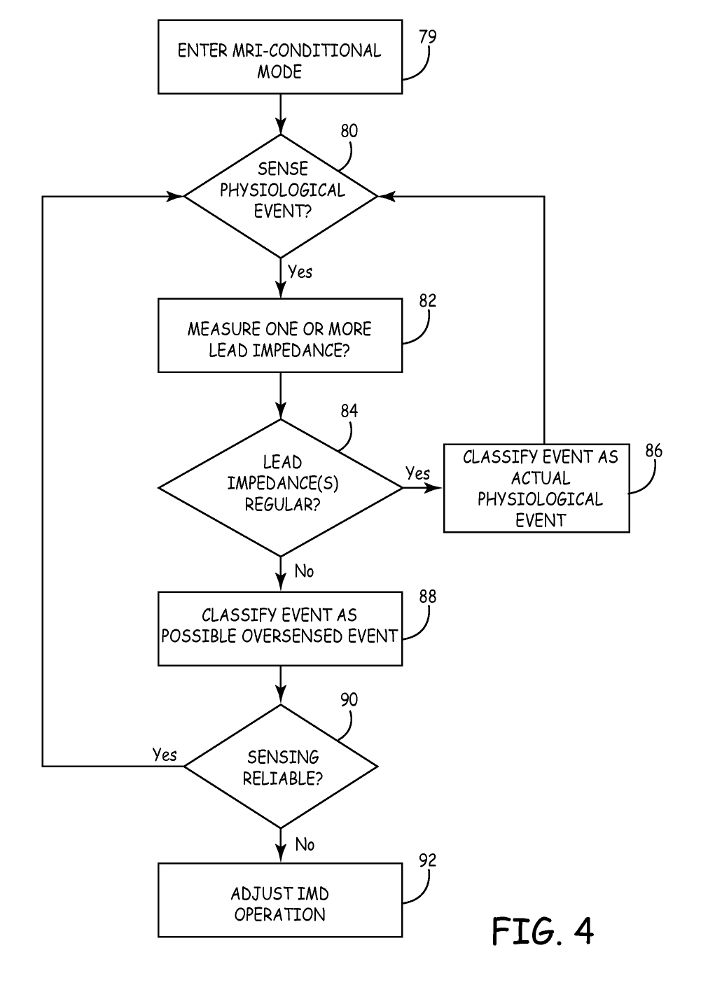

FIG. 4 is a flow diagram illustrating example operation of IMD 14 using lead impedance measurements to enhance sensing. Initially, IMD 14 enters the MRI-compatible operating mode (block 79). IMD 14 may be configured into the MRI-compatible operating mode prior to or immediately subsequent to entering environment 10. Although FIG. 4 is described as being performed while in the MRI-compatible operating mode, the techniques may be used in other operation modes, including a normal operating mode to assess the integrity of sensing when oversensing is suspected.

Sensing module monitors for a physiological event (block 80). When sensing module 62 senses physiological event (“Yes” branch of block 80), impedance measurement module 64 measures one or more lead impedances immediately subsequent to sensing the event (block 82). Impedance measurement module 64 may measure a single lead impedance or a series of two or more lead impedances. In one example, impedance measurement module 64 may begin to measure the one or more lead impedances within milliseconds or even microseconds of identifying the sensed event.

Impedance measurement module 64 includes circuitry to generate one or more impedance measurement signals and measure the impedances based on the one or more impedance measurement signals. Impedance measurement module 64 may, for example, generates one or more current pulses along an electrical path and measure voltages for each associated current pulse. In another example, impedance measurement module 64 may apply one or more voltages to an electrical path and measure currents for each applied voltage. Impedance measurement module 64 may then determine the lead impedances by dividing the voltages (either measured or sourced) by the currents (either sourced or measured, respectively).

Impedance measurement module 64 may determine whether the one or more lead impedances are regular (block 84). Impedance measurement module 64 may compare the one or more impedance measurements to an impedance range to determine whether the impedances are regular. In instances in which a series of lead impedances are measured, the impedance measurement module 64 may determine that the impedances are regular if all or an acceptable portion (e.g., 3 out of 4) of the impedance measurements are within the impedance range. Otherwise, impedance measurement module 64 may determine the impedances to be irregular. In another example, impedance measurement module 64 may determine the impedances to be regular if a morphology of the series of lead impedance measurements (i.e., amplitude of the impedance measurements over time) matches or correlates with a template morphology.

When impedance measurement module 64 determines that the lead impedances are regular (“Yes” branch of block 84), impedance measurement module 64 classifies the sensed event as an actual physiological event (block 86) and IMD 14 waits to sense another event (block 80). When impedance measurement module 64 determines that the lead impedances are not regular (“No” branch of block 84), impedance measurement module 64 classifies the sensed event as a possible oversensed event caused by the noise from interfering signal 11 (block 88). In this manner, impedance measurement module 64 determines whether the sensed events are actual sensed events or possible oversensed events based on the one or more impedance measurements subsequent to the sensed event.

Processor 60 (or impedance measurement module 64 in some instances) determines whether sensing is reliable (block 90). Processor 60 may determine whether sensing is reliable based on classifications of a number of consecutive sensed physiological events. Processor 60 may, for example, determine that sensing is not reliable when three of the last four sensed physiological events are classified as possible oversensed events. As another example, processor 60 may determine that sensing is not reliable when at least two sensed physiological events are classified as possible oversensed events within one second.

In response to determining that sensing is reliable (“Yes” branch of block 90), IMD 14 waits to sense another event (block 80). In response to determining that sensing is not reliable (“No” branch of block 90), processor 60 may further adjust operation of IMD 14 (block 92). Processor 60 may, for example, adjust operation of IMD 14 by changing sensing parameters, disabling sensing, changing pacing modes to a mode that is not dependent on sensing, withholding or delaying a therapy (e.g., tachyarrhythmia therapy), or the like.

The techniques described in this disclosure, including those attributed to one or more components of IMD 14, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, or other devices. The term “processor” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, SRAM, EEPROM, flash memory, magnetic data storage media, optical data storage media, or the like. The instructions may be executed to support one or more aspects of the functionality described in this disclosure.

Various examples have been described. In other instances, the techniques may be used to assess the integrity of a lead or a lead/device interface when oversensing is suspected. For example, to detect if a lead is not securely connected to a device or if a lead has experienced a conductor fracture, both of which may result in the device misinterpreting make/break connections as physiological signals. In yet other instances, the techniques may be applied during all sensed events. These and other examples are within the scope of the following claims.

1. A method comprising:
sensing a physiological event;
measuring one or more lead impedances subsequent to sensing the physiological event; and
classifying the sensed physiological event as an actual physiological event or a possible oversensed physiological event based on the one or more lead impedances.

2. The method of claim 1, further comprising:
identifying an irregularity in the one or more measured lead impedances; and
classifying the sensed physiological event as a possible oversensed physiological event in response to identifying the irregularity in the one or more measure lead impedances.

3. The method of claim 1, further comprising:
comparing each of the one or more lead impedances to a range of impedances range; and
classifying the sensed physiological event as a possible oversensed physiological event when an unacceptable portion of the one or more lead impedances is not within the range of impedances.

4. The method of claim 3, wherein classifying the sensed physiological event as a possible oversensed physiological event comprises classifying the sensed physiological event as a possible oversensed physiological event when at least one of the one or more lead impedances is not within the range of impedances.

5. The method of claim 3, wherein
measuring one or more lead impedances comprises measuring a series of two or more lead impedances, and
classifying the sensed physiological event as a possible oversensed physiological event comprises classifying the sensed physiological event as a possible oversensed physiological event when at least X of the lead impedances of the series are not within the range of impedances, wherein X is greater than one.

6. The method of claim 3, further comprising classifying the sensed physiological event as an actual physiological event when an acceptable portion of the lead impedances is within the range of impedances.

7. The method of claim 1, further comprising:
determining that sensing is unreliable using the classification of the sensed physiological event and a classification of one or more previously sensed physiological event; and
adjusting operation of the implantable medical device in response to determining that sensing is unreliable.

8. The method of claim 7, wherein determining that sensing is unreliable comprises determining that sensing is unreliable when the sensed physiological event and at least one of the one or more previously sensed physiological events are classified as possible oversensed events within a threshold period of time.

9. The method of claim 8, wherein adjusting operation of the implantable medical device in response to determining that sensing is unreliable comprises at least one of changing one or more sensing parameters, disabling sensing, adjusting a pacing mode to a mode that is not dependent on sensing, withholding delivery of a therapy and delaying delivery of a therapy.

10. The method of claim 1, wherein measuring one or more lead impedances comprises measuring a series of two or more lead impedances.

11. The method of claim 10, further comprising:
comparing a morphology of the series of lead impedances with a template morphology; and
classifying the sensed physiological event as a possible oversensed physiological event when the morphology of the series of lead impedances does not match the template morphology.

12. The method of claim 1, wherein
sensing a physiological event comprises sensing a physiological event on a first electrical path that includes a first pair of electrodes; and
measuring one or more lead impedances subsequent to sensing the physiological event comprises measuring one or more lead impedances on a second electrical path that includes at least one electrode that is different from the pair of electrodes of the first electrical path.

13. The method of claim 1, wherein
sensing a physiological event comprises sensing a physiological event on a first electrical path that includes a pair of electrodes; and
measuring one or more lead impedances subsequent to sensing the physiological event comprises measuring one or more lead impedances on the same electrical path on which the physiological event was sensed.

14. The method of claim 1, wherein measuring one or more lead impedances subsequent to sensing the physiological event comprises measuring the one or more lead impedances within microseconds of sensing the physiological event.

15. An implantable medical device comprising
a sensing module that senses a physiological event;
an impedance measurement module that measures one or more lead impedances subsequent to the sensing module sensing the physiological event and classifies the sensed physiological event as an actual physiological event or a possible oversensed physiological event based on the one or more lead impedance measurements.

16. The device of claim 15, wherein the impedance measurement module identifies an irregularity in the one or more measured lead impedances and classifies the sensed physiological event as a possible oversensed physiological event in response to identifying the irregularity in the one or more measure lead impedances.

17. The device of claim 15, wherein the impedance measurement module compares each of the one or more lead impedances to a range of impedances range and classifies the sensed physiological event as a possible oversensed physiological event when an unacceptable portion of the one or more lead impedances is not within the range of impedances.

18. The device of claim 17, wherein the impedance measurement module classifies the sensed physiological event as a possible oversensed physiological event when at least one of the one or more lead impedances is not within the range of impedances.

19. The device of claim 17, wherein the impedance measurement module measures a series of two or more lead impedances and classifies the sensed physiological event as a possible oversensed physiological event when at least X of the lead impedances of the series are not within the range of impedances, wherein X is greater than one.

20. The device of claim 17, wherein the impedance measurement module classifies the sensed physiological event as an actual physiological event when an acceptable portion of the lead impedances is within the range of impedances.

21. The device of claim 15, further comprising a processor that determines that sensing is unreliable using the classification of the sensed physiological event and a classification of one or more previously sensed physiological event and adjusts operation of the implantable medical device in response to determining that sensing is unreliable.

22. The device of claim 21, wherein the processor determines that sensing is unreliable when the sensed physiological event and at least one of the one or more previously sensed physiological events are classified as possible oversensed events within a threshold period of time.

23. The device of claim 22, wherein the processor adjusts operation of the implantable medical device in response to determining that sensing is unreliable by at least one of changing one or more sensing parameters, disabling sensing, adjusting a pacing mode to a mode that is not dependent on sensing, withholding delivery of a therapy and delaying delivery of a therapy.

24. The device of claim 15, wherein the impedance measurement module measures a series of two or more lead impedances.

25. The device of claim 24, wherein the impedance measurement module compares a morphology of the series of lead impedances with a template morphology and classifies the sensed physiological event as a possible oversensed physiological event when the morphology of the series of lead impedances does not match the template morphology.

26. The device of claim 15, wherein the impedance measurement module senses a physiological event on a first electrical path that includes a first pair of electrodes and measures the one or more lead impedances on a second electrical path that includes at least one electrode that is different from the pair of electrodes of the first electrical path.

27. The device of claim 15, wherein the impedance measurement module senses a physiological event on a first electrical path that includes a pair of electrodes and measures one or more lead impedances on the same electrical path on which the physiological event was sensed.

28. The device of claim 15, wherein the impedance measurement module measures the one or more lead impedances within microseconds of sensing the physiological event.

29. An implantable medical device comprising:
means for sensing a physiological event;
means for measuring one or more lead impedances subsequent to sensing the physiological event; and
means for classifying the sensed physiological event as an actual physiological event or a possible oversensed physiological event based on the one or more lead impedances.

30. The device of claim 29, further comprising:
means for comparing each of the one or more lead impedances to a range of impedances range;
wherein the classifying means classify the sensed physiological event as a possible oversensed physiological event when an unacceptable portion of the one or more lead impedances is not within the range of impedances.

31. The device of claim 30, wherein the classifying means classifies the sensed physiological event as an actual physiological event when an acceptable portion of the lead impedances is within the range of impedances.

32. The device of claim 29, further comprising:
means for determining that sensing is unreliable using the classification of the sensed physiological event and a classification of one or more previously sensed physiological event; and
means for adjusting operation of the implantable medical device in response to determining that sensing is unreliable.

33. The device of claim 32, wherein the determining means determines that sensing is unreliable when the sensed physiological event and at least one of the one or more previously sensed physiological events are classified as possible oversensed events within a threshold period of time.

34. The device of claim 29, wherein the measuring means measures a series of two or more lead impedances.

35. The device of claim 34, further comprising:
means for comparing a morphology of the series of lead impedances with a template morphology;
wherein the classifying means classifies the sensed physiological event as a possible oversensed physiological event when the morphology of the series of lead impedances does not match the template morphology.

36. A computer-readable medium comprising instructions that, when executed by a processor, cause the processor to:
sense a physiological event;
measure one or more lead impedances subsequent to sensing the physiological event; and
classify the sensed physiological event as an actual physiological event or a possible oversensed physiological event based on the one or more lead impedances.

37. The computer-readable medium of claim 36, further comprising instructions that, when executed by the processor, cause the processor to:
compare each of the one or more lead impedances to a range of impedances range; and
classify the sensed physiological event as a possible oversensed physiological event when an unacceptable portion of the one or more lead impedances is not within the range of impedances.

38. The computer-readable medium of claim 36, wherein a series of lead impedances are measured, the computer-readable medium further comprising instructions that, when executed by the processor, cause the processor to:
compare a morphology of the series of lead impedances with a template morphology; and
classify the sensed physiological event as a possible oversensed physiological event when the morphology of the series of lead impedances does not match the template morphology.

39. The computer-readable medium of claim 36, further comprising instructions that, when executed by the processor, cause the processor to:
determine that sensing is unreliable using the classification of the sensed physiological event and a classification of one or more previously sensed physiological event; and
adjust operation of the implantable medical device in response to determining that sensing is unreliable.
